(19) United States
(12) Patent Application Publication
(10) Pub. No.: US 2006/0282030 A1
(43) Pub. Date: Dec. 14, 2006

(54) System for forming a structure having load-bearing capabilities

(76) Inventors: James C. Martin, Baldwin City, KS (US); Larry R. DeCamp, Lawrence, KS (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/150,404

(22) Filed: Jun. 10, 2005

(51) Int. Cl. A61F 5/00

(52) U.S. Cl. 602/8

(57) ABSTRACT

A system for forming a structure having load-bearing capabilities is provided. This system includes a material capable of conforming to a desired shape and an activator that includes a pre-measured amount of fluid. Both the material and the activator are contained within an activator-impervious sealed pouch. The pouch is clamped or sealed so as to isolate the conformable material from the activator by creating two separated areas within the pouch. In use, a device clamping the pouch is removed or an internal frangible seal of the pouch is broken so as to allow the activator to be mixed with the conformable material. Following this, the pouch is opened, and the activated material is removed. The activated material then may be applied to a surface where it hardens and forms a structure having load-bearing capabilities.

### CROSS-REFERENCE TO RELATED APPLICATIONS

Not Applicable.

### STATEMENT REGARDING FEDERALLY SPONSORED RESEARCH OR DEVELOPMENT

Not applicable.

### BACKGROUND OF THE INVENTION

A system for forming a structure having load-bearing capabilities is provided. More specifically, this system is self-contained. Both the material used to form the structure and the fluid for initiating setting of the material are contained within the same sealed pouch.

Orthopedic bandages available today for making casts include open-weave fabric coated with a prepolymer, such as a polyurethane prepolymer. When the bandage is to be used, it is removed from a package and dipped in a bucket of water or placed under running water to initiate setting. Once the bandage is removed from the water, it is applied to a patient, perhaps over a stockinette and cast padding. The bandage then will set so as to immobilize that portion of the patient's body to which it is applied.

One disadvantage with the current method of activating such a bandage is that it is difficult to obtain the necessary amount of water needed to polymerize the prepolymer while not over wetting the bandage. Many times the bandage being placed on a patient holds excess water even after having been wrung out. Placing a bandage having excess water near a patient's skin can cause skin maceration and sloughing of the skin. For certain prepolymers, excessive water on the bandage also can cause an overly exothermic polymerization reaction, which can burn the patient's skin. Further, if less than the necessary amount of water is used, the prepolymer will not polymerize in a desired period of time, and the bandage may not sufficiently harden so as to immobilize that portion of the patient's body to which it is applied.

Another disadvantage of the orthopedic cast bandages currently available is that one must make sure to have a water source available at the location where the bandage is to be activated. Needing a water source limits the portability of these bandages. Using these bandages can be inconvenient when trying to apply the bandages in remote locations, such as in underdeveloped areas that do not have running water. In addition, these conventional bandages, which need a water source to activate them, are especially difficult for deployed military units to use.

Another disadvantage with traditional orthopedic cast bandages is that the process for initiating setting of these materials is often messy and time consuming. After a bandage is activated in a bucket of water, the water becomes dirty or contaminated. Yet, many times this contaminated water is reused to activate further bandages. In addition, when a bandage is over wetted, it will hold excess water even after having been wrung out, and this excess water can drip from the bandage causing a mess. The water may mix with the prepolymer, and the mixture may drip on the floor, on a person's clothes, on a patient's skin, or into the pipes of a sink so as to clog plumbing. Such dripping bandages can be even messier if colored prepolymers and/or fabrics are used. Still further, it is time consuming to make sure the bandage is wet sufficiently, wring the bandage out, and clean up any mess made by the dripping bandage.

For the foregoing reasons, a better system for activating an orthopedic bandage is needed. This system should be more efficient to use and create less of a mess. Further, this system should be able to be used for other applications besides orthopedic applications so that structures having load-bearing capabilities can be prepared in a neat and efficient manner for a variety of applications.

### BRIEF SUMMARY OF THE INVENTION

It is an object of the present invention to provide an easily transportable system for forming a structure having load-bearing capabilities so that it can be used conveniently.

According to the present invention, the foregoing and other objects are achieved by a system for forming a structure having load-bearing capabilities. This system includes a material capable of conforming to a desired shape and an activator that includes a pre-measured amount of fluid, such as water. The conformable material includes a substrate with resin thereon. Both the conformable material and the activator are contained within an activator-impervious sealed pouch. The pouch is clamped or sealed so as to isolate the conformable material from the activator by creating two separated areas within the pouch. In use, a clip is removed from the pouch or an internal frangible seal of the pouch is broken so as to allow the activator to be mixed with the conformable material to initiate setting. Following this, the pouch is opened, and the activated material is removed. The activated material then may be applied to a surface where it hardens and forms a structure having load-bearing capabilities.

In another embodiment of the present invention, the conformable material is a substrate with an activator thereon. In this embodiment, the conformable material with activator is kept in one separated area of the sealed pouch while a resin is kept in the other separated area of the pouch. In use, the structure isolating one area of the sealed pouch from the other is removed to allow the resin to mix with the conformable material and activator in order to form a structure that hardens into a load-bearing structure.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

In the accompanying drawings, which form a part of the specification and are to be read in conjunction therewith, and in which like reference numerals are used to indicate like parts in the various views:

FIG. 1 is a perspective view of a sealed pouch of a preferred embodiment of the present invention that encloses water and a conformable material and has a clip that clamps the pouch so as to isolate the water from the conformable material;

FIG. 2 is a plan view of the sealed pouch shown in FIG. 1 with the conformable material and the water shown in broken lines;

FIG. 3 is a side elevational view of the sealed pouch shown in FIG. 1;

FIG. 4 is an enlarged end view of the portion of the pouch and clip within captured region (4) of FIG. 3;

FIG. 5 is a plan view of the sealed pouch shown in FIG. 2 with the clip removed and the water flowing into the chamber of the conformable material;

FIG. 6 is a plan view of the pouch shown in FIG. 2 that has been torn open and the conformable material has been partially unwound;

FIG. 7 is a horizontal cross-sectional view of the conformable material taken along line 7-7 of FIG. 6;

FIG. 8 is a plan view similar to FIG. 6 showing an alternative embodiment of the present invention;

FIG. 9 is a perspective view of a kit that includes a pouch according to the present invention, gloves, a stockinette, and a roll of cast padding in a container;

FIG. 10 is a perspective view of a sealed pouch of an alternative embodiment of the present invention;

FIG. 11 is a side elevational view of the sealed pouch shown in FIG. 10;

FIG. 12 is a vertical cross-sectional view of the pouch taken along line 12-12 of FIG. 10;

FIG. 13 is a vertical cross-sectional view of the pouch shown in FIG. 10 with a person applying pressure to the water in the pouch; and

FIG. 14 is a vertical cross-sectional view of the pouch shown in FIG. 10 with the frangible seal being broken from the pressure applied to the water in the pouch.

### DETAILED DESCRIPTION OF PREFERRED EMBODIMENT

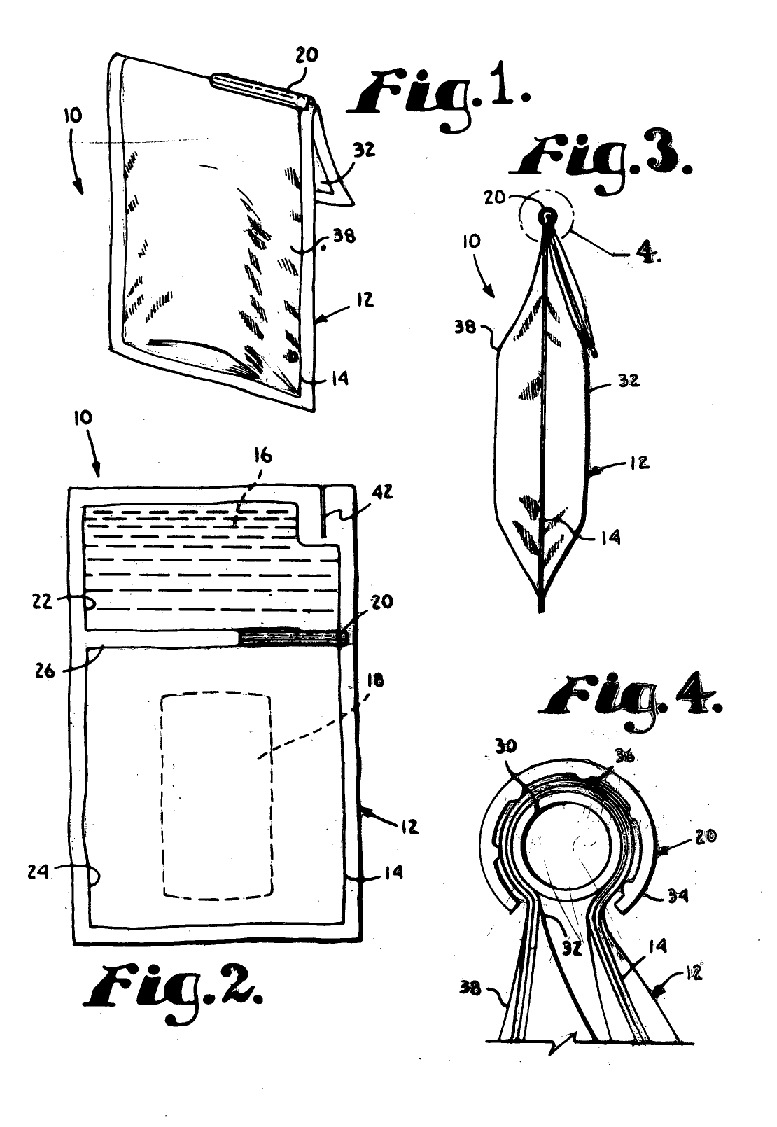

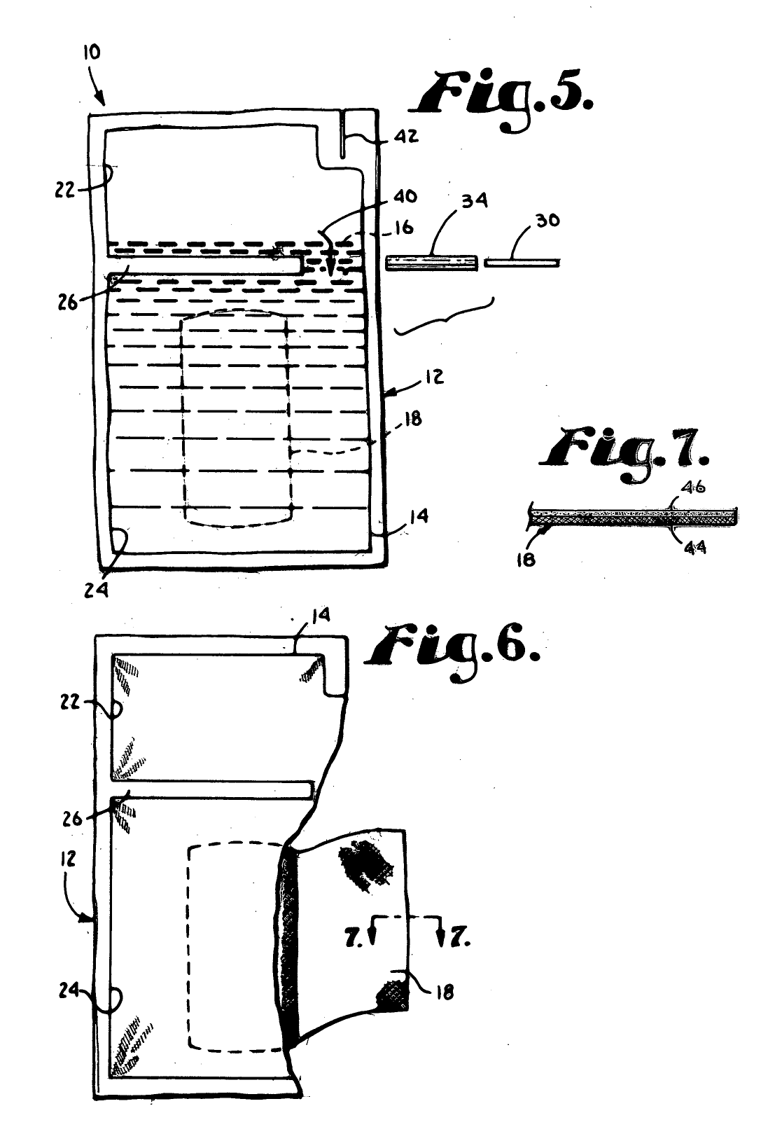

Referring to FIG. 1, a system for forming a structure having load-bearing capabilities is shown, and this system is broadly designated by reference numeral 10. System 10 includes a pouch 12 having a water-impervious seal 14 around its edge. As best shown in FIG. 2, pouch 12 encloses water 16 and a resin-impregnated material 18 that is capable of conforming to a desired shape. As shown in FIGS. 1-4, a clip 20 clamps pouch 12 so as to isolate water 16 from material 18 by creating two separated areas 22 and 24 within pouch 12 until hardening of material 18 is desired. As best shown in FIG. 2, pouch 12 has a seal 26 between separated areas 22 and 24 that partially separates these areas so that clip 20 must only clamp part of pouch 12 in order to isolate material 18 from water 16. Area 22 contains a pre-measured amount of water. This amount of water in area 22 is the precise amount needed to activate material 18 in a desired amount of time.

Clip 20 is best shown in FIG. 4. It includes a tubular piece 30 that is placed next to a first side 32 of pouch 12 and a semi-tubular piece 34 having pressure enhancers 36 that is placed next to a second side 38 of pouch 12. Tubular piece 30 is received by semi-tubular piece 34 so as to clamp sides 32 and 38 of pouch 12 together. Pressure enhancers 36 help to press sides 32 and 38 of pouch 12 together so as to isolate water 16 from material 18.

Tubular piece 30 and semi-tubular piece 34 of clip 20 also are shown in FIG. 5 removed from pouch 12. When clip 20 is removed from pouch 12, water 16 is able to flow into area 24, as designated by arrow 40 in FIG. 5, so as to initiate the setting of material 18.

Pouch 12 has a line of weakness 42, which is shown in FIGS. 2 and 5, for directing the location at which the pouch is opened and providing a line for tearing the pouch so that the wetted material can be removed. As best shown in FIG. 6, pouch 12 includes a roll of resin-impregnated material 18. Material 18 includes a knitted fiberglass fabric 44 impregnated with a polyurethane prepolymer 46, as best shown in FIG. 7.

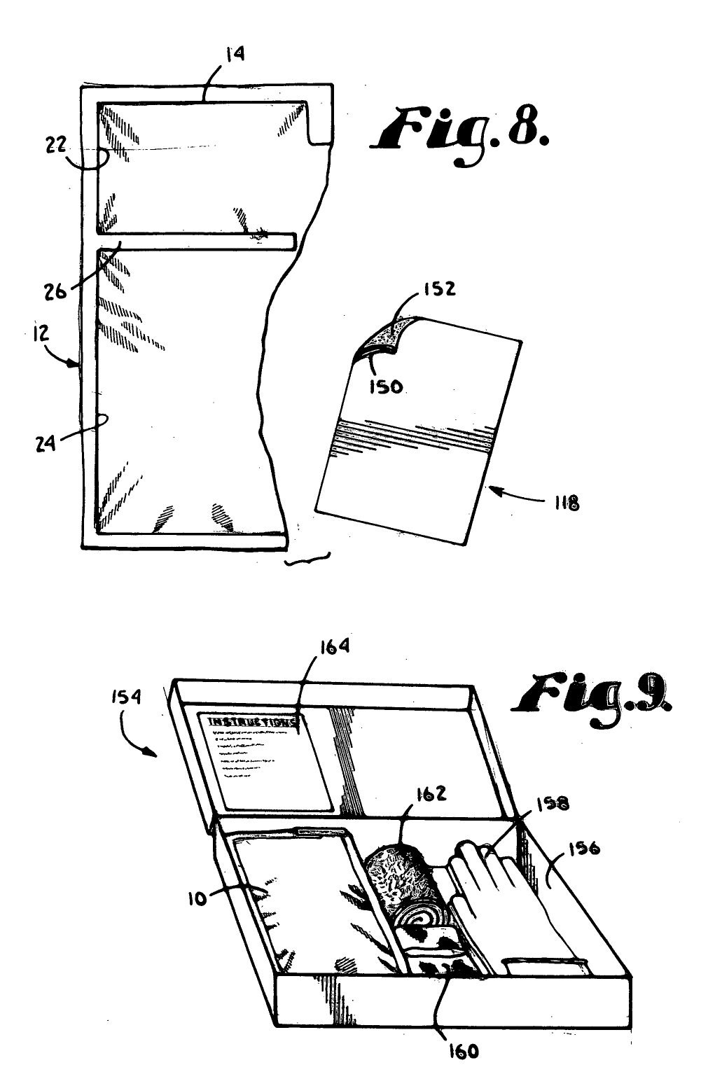

An alternative embodiment of the material capable of conforming to a desired shape is shown in FIG. 8 and is broadly designated by reference numeral 118. Conformable material 118 is a sheet made of a flexible non-porous plastic substrate 150 with a polyurethane prepolymer 152 thereon.

Referring to FIG. 9, a portable kit that includes system 10 is shown and is broadly designated by reference numeral 154. A container 156 holds system 10 and supplies to be used when applying the conformable material in system 10 as a bandage to a patient for an orthopedic use. More specifically, the conformable material may be a bandage that forms a cast when hardened. The supplies held by container 156 include gloves 158, a stockinette 160, a roll of cast padding 162, and an instruction sheet 164.

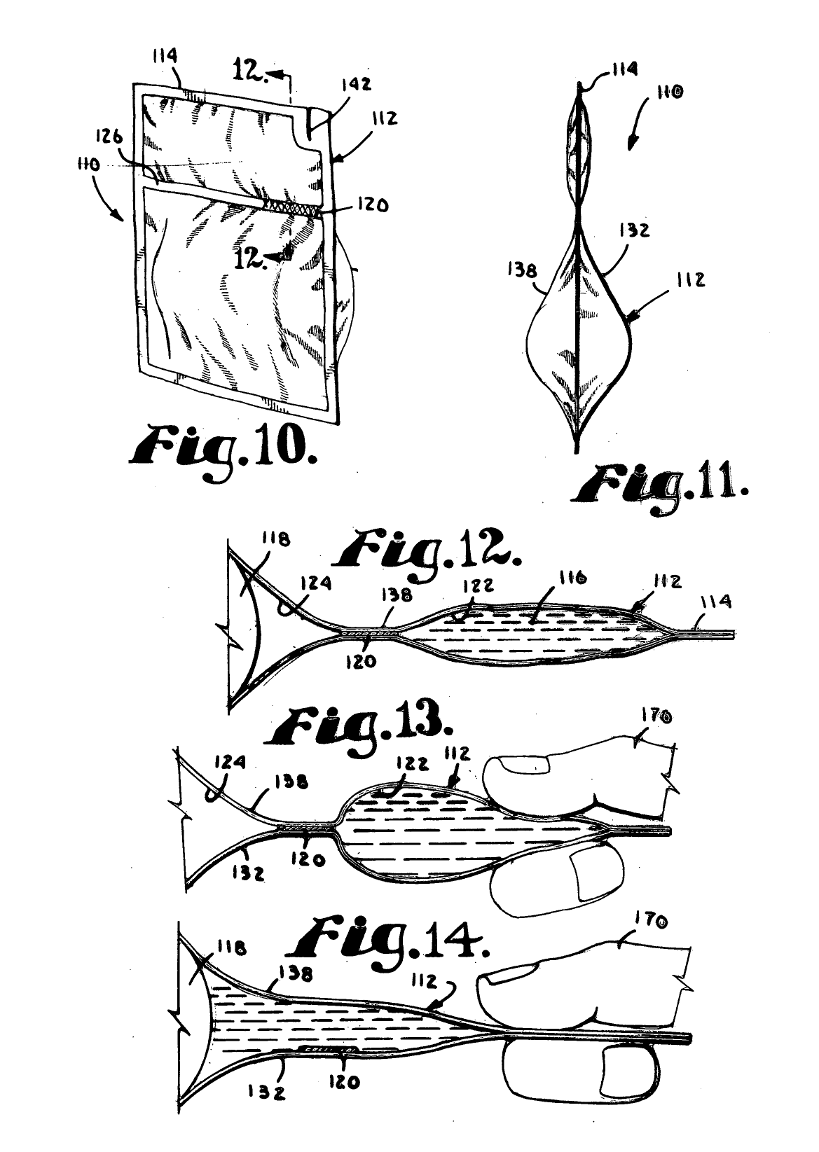

An alternative embodiment of the system of the present invention is shown in FIGS. 10 and 11 and is broadly designated by reference numeral 110. System 110 includes pouch 112 having a water-impervious seal 114 around its edge. Pouch 112 encloses water 116 and a resin-impregnated material 118 that is capable of conforming to a desired shape, as best shown in FIGS. 12-14. Pouch 112 has sides 132 and 138. A frangible seal 120 seals sides 132 and 138 together so as to subdivide pouch 112 and isolate water 116 from material 118 by creating two separated areas 122 and 124 within pouch 112 until hardening of material 118 is desired. Pouch 112 has a sturdy seal 126 between separated areas 122 and 124 that partially separates these areas so that frangible seal 120 must only seal part of pouch 112 in order to isolate material 118 from water 116. Seals 114 and 126 are not as easily broken as frangible seal 120. Frangible seal 120 may be broken upon receiving water pressure created by pressure from one's fingers being placed on area 122, which contains a pre-measured amount of water. This amount of water in area 122 is the precise amount needed to activate material 118 in a desired amount of time.

When the embodiment of FIG. 1 is in use, clip 20 is removed from pouch 12 so as to allow water 16 to enter into area 24 so as to initiate activation of conformable material 18. Area 24 of pouch 12 is squeezed to push the air surrounding material 18 into area 22, which in turn will cause water 16 from area 22 to move into area 24. Air opens the passageway between areas 22 and 24. Then, water 16 immediately exits area 22 and contacts material 18 in area 24. By squeezing area 24 of pouch 12 rather than area 22 allows the passageway to open quicker so that water 16 can reach material 18 with the most efficiency. Once water 16 is sufficiently mixed with conformable material 18 so as to initiate setting of this material, pouch 12 is opened by tearing it along line of weakness 42, and the activated material is removed. One side of the pouch may be torn off completely by extending the tear in a line along the line of weakness, if desired, for easy access to the activated material. The activated material then may be applied to a surface where it hardens and forms a structure having load-bearing capabilities. Typically, the activated material will harden in about 2 minutes to about 1 hour after receiving the activator. Preferably, the activated material hardens in about 2-4 minutes after receiving the activator.

When the embodiment of FIG. 10 is in use, pressure is placed on area 122 of pouch 112 by fingers 170 so as to cause water 116 to break frangible seal 120, as best shown in FIGS. 13 and 14. Once frangible seal 120 is broken, water 116 can enter into area 124 so as to initiate activation of conformable material 118. Area 122 of pouch 12 may continue to be squeezed in order to move water 116 into area 124. As discussed previously, once water 116 is sufficiently mixed with material 118 so as to initiate setting of this material, pouch 112 is opened by tearing it along a line of weakness 142, and the activated material is removed.

By having a system that includes a conformable material and water for activating the material, neither a bucket of water nor running water is needed. Thus, contamination of the water used for activation and plumbing clogs are avoided. Further, if an activator other than water is used, an external supply source for that activator is not needed.

The system of the present invention also provides a controlled environment for mixing the activator with the conformable material. Because the activator and the conformable material are mixed within a sealed pouch, contaminants cannot touch the conformable material during the mixing process.

Preferably, system 10 is part of a kit that includes gloves 158. Preferably, gloves 158 are disposable gloves. Gloves 158 may be worn by a person applying the activated material to a surface so as to avoid having to touch it. If the activated material is for orthopedic use, preferably, the kit also contains a stockinette 160 for placing on a patient and a roll of cast padding 162 for placing around stockinette 160 before applying the activated material to the cast padding. Putting all the supplies to be used with system 10 in a container allows for easy transportability and availability of materials.

Because the system of the present invention is self-contained and easily transportable, it is useful in remote locations. It may be used on ambulances, in underdeveloped areas that do not have running water, and by deployed military units.

While water 16 is a preferred material for initiating setting of many conformable materials, other activating materials may be used depending upon the material being activated, and additives may be added to the activating material to enhance performance. In the broadest first embodiment of the invention, area 22 contains an activator.

The activator includes a pre-measured amount of fluid. This pre-measured amount should be sufficient to ensure full activation of the material in a desired time frame so that the material will harden and form a structure with load-bearing capabilities. Further, this amount should not be excessive so as to cause the activated material to drip as it is being applied or create wetness next to a patient's skin as it is being applied.

The fluid in the activator may be a liquid or a gas. The fluid should compliment the material being activated so as to be able to activate the material after the material is exposed to the fluid. For many desired materials, water is a preferred fluid for use as an activator. For other materials, such as materials for boats made from fiberglass and polyester resin, methylethyl ketone peroxide is a desirable fluid for activation.

Other components may be included as part of the activator in addition to the activating fluid. Such other components include a colorant, a lubricant, an antimicrobial agent, a fragrance, and/or a catalyst. The colorant allows structures of different colors to be formed without having to manufacture the conformable material in different colors. By merely coloring the material upon activation, the need to make and store the conformable material in a variety of colors is avoided.

Many times the activated conformable material is sticky and difficult to use, and it may stick to the gloves of a person applying it. It also may have a rough surface once it hardens. Therefore, it is desirable to add a lubricant to the fluid in an attempt to keep the material from sticking to one's gloves. Further, adding a lubricant to the fluid creates a hardened material with a smoother surface, which helps to prevent people from snagging their clothing on the surface of the hardened material. The lubricant may be, but is not limited to, silicon and/or sodium lauryl sulfate.

The addition of an antimicrobial agent to the activator increases the shelf life of certain activating fluids. Still further, it may help to prevent skin maceration and sloughing if the activated material is used as an orthopedic bandage.

In certain circumstances, it may be desirable to include a fragrance as part of the activator. This fragrance may be able to mask any odor that the conformable material or the fluid may have. A fragrance also may be useful for creating products with a variety of desirable scents.

A catalyst may be added to the activating fluid to promote and/or increase the speed of the hardening of the material. For instance, if a knitted fiberglass fabric substrate is impregnated with a polyurethane prepolymer resin, a catalyst may help promote the polymerization reaction as the water reacts with the prepolymers.

While a water-impervious seal 14 for pouch 12 is desired when water is the activator, if an activator other than water is used, then the seal of the pouch should be impervious to that activator. In the broadest first embodiment of the present invention, the pouch has an activator-impervious seal so as to keep whatever activator is being used from escaping from the pouch before it is allowed to activate the conformable material. Such a seal also keeps an activator that is in the environment outside of the pouch, such as moisture in the air, from prematurely contacting the conformable material so as to initiate its activation. In many cases, a heat seal is used.

In addition, the pouch should be made of a material that is activator-impervious and that remains activator-impervious when exposed to hot and cold temperatures. Further, the pouch should remain activator-impervious when transported at high altitudes, such as in airplanes. In many circumstances, the pouch is made of polyester film with an aluminum foil moisture barrier and a polyethylene heat sealant coating.

Materials 18 and 118 are non-fluid and should be capable of conforming to a desired shape and then harden and form a structure having load-bearing capabilities after they are activated. Other substrates may be used in place of knitted fiberglass fabric 44 or plastic substrate 150 for receiving polyurethane prepolymer. Preferably, for most applications, flexible substrates are used. Also, the substrate is made from a non-fluid material as described in more detail below. Other resins, whether polymeric or not, that harden upon exposure to a particular gas or liquid so as to form a structure with the substrate having load-bearing capabilities may be used in place of polyurethane prepolymers 46 and 152. A catalyst may be added to the resin so as to help promote the reaction between the activator and the resin.

Other open-matrix substrates may be used in place of knitted fiberglass fabric 44. These open-matrix substrates are impregnated with a structural resin that hardens upon exposure to a fluid. Preferably, the open-matrix substrate has a knitted or woven configuration.

Other non-porous substrates may be used in place of plastic substrate 150. With these non-porous substrates, a structural resin that hardens upon exposure to a fluid is placed on the substrate.

The open-matrix or non-porous substrates are non-fluid materials (i.e., a solid and not a liquid or a gas) having structural integrity and may be made from a variety of materials including, but not limited to, metal, plastic, fiberglass, thermoset resins, cotton, or synthetic fibers. The substrate may be made into a variety of configurations including, but not limited to, a knit, a weave, a foam, a needle-punched felt, or a spun-bonded laminate.

In an alternate embodiment of the present invention, a substrate is coated or impregnated with an activator and is kept in one area of the sealed pouch while a resin is isolated from the activator-coated material by being placed in the other area of the sealed pouch. When the structure for isolating one area of the pouch from the other is removed, the resin is mixed with the activator-coated material, and the substrate becomes coated and/or impregnated with activated resin. The resin is activated at the same time that it is mixed with the substrate so as to form a structure that hardens within a few minutes and has load-bearing capabilities.

Clip 20 may be any device that is able to clamp sides 32 and 38 of pouch 12 together so as to isolate the activator from the conformable material. Preferably, the device includes a tubular piece and a semi-tubular piece, as shown in FIGS. 2 and 5. These pieces may, but need not, be made of plastic. It is also contemplated that for certain resins and activators a spring-loaded clip will be sufficient to isolate these materials until activation is desired. Alternatively, a frangible seal 120 that subdivides the sealed pouch into two areas may be used in place of clip 20, as shown in FIGS. 10-14. In the broadest embodiment of the present invention, any structure that is able to isolate the material in one area of the pouch from the fluid, whether a resin or an activator, in the other area of the pouch may be used.

In addition to being conformable, preferably, the material is able to adhere to itself and to other surfaces such as metal, plastic, fiberglass, polymers, cotton padding, or fabric. This allows the system of the present invention to be used for a variety of applications. It may be used when structural stiffness is desired without having to use auxiliary materials or devices. It may be used to form a desired shape, may be applied to a surface, or may be used to create a mold. An appropriate substrate and resin for making a conformable material should be chosen based upon the application for which the material is to be used.

The system of the present invention may be used as a system for forming orthopedic casts and splints. This allows load-bearing structures to be formed so as to immobilize a desired area of a patient's body. The system also may be used to form other load-bearing structures such as prosthesis, orthotics, or other structures requiring complex form replication. It also may be useful for arts and crafts applications.

The system of the present invention also may be used for fluid pressure containment so as to repair a structure. For instance, this system may be used to provide material for wrapping pipes, repairing mufflers, patching farm tanks, and sealing pressure vessels.

Other applications for the system of the present invention include, but are not limited to, repairing the structural surfaces of boats, airplanes, and automobiles. The activator may contain a colorant so that the color of the material being used to repair the surface matches the color of the surface being repaired.

From the foregoing it will be seen that this invention is one well adapted to attain all ends and objectives hereinabove set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

We claim:

1. A system for forming a structure having load-bearing capabilities, said system comprising:
a non-fluid material capable of conforming to a desired shape, wherein said material hardens and forms a structure having load-bearing capabilities after being activated;
a pre-measured amount of fluid;
an activator-impervious sealed pouch, wherein said material and said fluid are within said sealed pouch; and
isolating structure that creates two separated areas within said pouch for keeping said material separated from said fluid until hardening of said material is desired.

2. The system of claim 1 wherein said isolating structure is a device that clamps said pouch so as to isolate said material from said fluid.

3. The system of claim 1 wherein said isolating structure is a frangible seal that subdivides said sealed pouch into two areas so as to isolate said material from said fluid.

4. The system of claim 1 wherein said fluid is comprised of a resin and said material has an activator for said resin thereon.

5. The system of claim 1 wherein said material has a resin thereon and said fluid is comprised of an activator for said resin.

6. The system of claim 5 wherein said fluid is a gas.

7. The system of claim 5 wherein said fluid is a liquid.

8. The system of claim 7 wherein said activator is methylethyl ketone peroxide.

9. The system of claim 7 wherein said activator is water.

10. The system of claim 2 wherein said pouch has a partially sealed area between said separated areas so that said device must only clamp part of said pouch in order to isolate said material from said fluid.

11. The system of claim 2 wherein said device is a clip.

12. The system of claim 1 wherein said pouch has a line of weakness in said seal for opening said pouch so as to be able to remove said activated material.

13. The system of claim 1 wherein said material is an open-matrix substrate impregnated with a resin and said fluid is comprised of an activator for said resin.

14. The system of claim 13 wherein said resin is comprised of prepolymers that polymerize upon exposure to water.

15. The system of claim 14 wherein said resin is comprised of a polyurethane prepolymer.

16. The system of claim 13 wherein said open-matrix substrate is comprised of knitted fiberglass fabric.

17. The system of claim 1 wherein said material is comprised of a substrate and a resin and said substrate is selected from the group consisting of metal, plastic, fiberglass, thermoset resins, cotton, and synthetic fibers.

18. The system of claim 17 wherein said substrate is configured as a knit, a weave, a foam, a needle-punched felt, or a spun-bonded laminate.

19. The system of claim 2 wherein said device is comprised of a tubular piece that is placed on a first side of said pouch and a semi-tubular piece placed on a second side of said pouch wherein said tubular piece is received by said semi-tubular piece so as to clamp said pouch.

20. The system of claim 19 wherein said semi-tubular piece has pressure enhancers thereon that are placed against said second side of said pouch.

21. A method of forming a structure having load-bearing capabilities, comprising:
providing a material capable of conforming to a desired shape and an activator comprised of a pre-measured amount of fluid, wherein said material and said activator are in an activator-impervious sealed pouch and wherein a device clamps said pouch so as to isolate said material from said activator by creating two separated areas within said pouch;
removing said device from said pouch;
mixing said activator with said material;
opening said pouch;
removing said activated material from said pouch; and
applying said activated material to a surface, wherein said activated material hardens and forms a structure having load-bearing capabilities.

22. The method of claim 21 wherein said activator is mixed with said material by applying pressure to a first separated area containing said material, wherein air in said first area opens a passageway between said two separated areas after said device is removed so as to cause said activator from said second separated area to enter said first area.

23. The method of claim 21 wherein said material hardens in about 2 minutes to about 1 hour after receiving said activator.

24. The method of claim 21 wherein said pouch has a line of weakness in said seal and wherein said pouch is opened by tearing said pouch at said line of weakness.

25. The method of claim 21 wherein said surface is comprised of metal, plastic, fiberglass, cotton padding, or fabric and wherein said activated material adheres to said surface.

26. A kit for forming a structure having load-bearing capabilities for application to a surface, said kit comprising:
a material that is capable of conforming to a desired shape, wherein said material is comprised of a resin-impregnated substrate and wherein said material hardens and forms a structure having load-bearing capabilities after being activated;
an activator comprised of a pre-measured amount of fluid said amount being sufficient to harden said material;
an activator-impervious sealed pouch, wherein said material and said activator are within said sealed pouch;
isolating structure that creates two separated areas within said pouch for keeping said material separated from said activator until hardening of said material is desired;
gloves for use by a person applying said material to said surface; and
an instruction sheet.

27. The kit of claim 26, further comprising:
cast padding for placing on a patient before applying said material thereto; and
a stockinette for placing on said patient beneath said cast padding.

28. A system for forming a structure having load-bearing capabilities, said system comprising:
a non-fluid material capable of conforming to a desired shape, wherein said material hardens and forms a structure having load-bearing capabilities after being activated;
a pre-measured amount of fluid;
an activator-impervious sealed pouch, wherein said material and said fluid are within said sealed pouch; and
means for isolating said material from said fluid by creating two separated areas within said pouch until hardening of said material is desired.

29. The system of claim 28 wherein said means for isolating said material from said fluid is a device that clamps said pouch so as to isolate said material from said fluid.

30. The system of claim 28 wherein said means for isolating said material from said fluid is a frangible seal that subdivides said sealed pouch into two areas so as to isolate said material from said fluid.

31. A system for forming an orthopedic cast bandage, said system comprising:
a material capable of conforming to a desired shape, wherein said material is comprised of a resin-impregnated substrate and wherein said material hardens and forms a structure having load-bearing capabilities after being activated;
an activator comprised of a pre-measured amount of fluid, said amount being sufficient to harden said material;
an activator-impervious sealed pouch, wherein said material and said activator are within said sealed pouch; and
a device that clamps said pouch so as to isolate said material from said activator by creating two separated areas within said pouch until hardening of said material is desired.

32. The system of claim 31 wherein said fluid is water.

33. The system of claim 31 wherein said activator further comprises a colorant.

34. The system of claim 31 wherein said activator further comprises a lubricant.

35. The system of claim 31 wherein said activator further comprises an antimicrobial agent.

36. The system of claim 31 wherein said activator further comprises a fragrance.

37. The system of claim 31 wherein said activator further comprises a catalyst.

38. The system of claim 31 wherein said activator further comprises a colorant, a lubricant, and an antimicrobial agent.

39. The system of claim 38 wherein said activator further comprises a catalyst.

40. The system of claim 31 wherein said pouch has a partially sealed area between said separated areas so that said device must only clamp part of said pouch in order to isolate said material from said activator.

41. The system of claim 31 wherein said material is an open-matrix substrate impregnated with a resin comprised of prepolymers that polymerize upon exposure to water.

42. The system of claim 41 wherein said resin is comprised of a polyurethane prepolymer.

43. The system of claim 42 wherein said open-matrix substrate is comprised of knitted fiberglass fabric.

44. The system of claim 31 wherein said device is comprised of a tubular piece that is placed on a first side of said pouch and a semi-tubular piece placed on a second side of said pouch wherein said tubular piece is received by said semi-tubular piece so as to clamp said pouch.
